(19) United States
(12) Patent Application Publication
(10) Pub. No.: US 2014/0160262 A1
(43) Pub. Date: Jun. 12, 2014

(54) LENSLESS RETINAL CAMERA APPARATUS AND METHOD

(71) Applicants: Mihai Busuioc, Rock Hill, NY (US); R. Theodore Smith, Demarest, NJ (US)

(72) Inventors: Mihai Busuioc, Rock Hill, NY (US); R. Theodore Smith, Demarest, NJ (US)

(21) Appl. No.: 13/997,325

(22) PCT Filed: Dec. 22, 2011
(86) PCT No.: PCT/US11/66858

§ 371 Date: Feb. 14, 2014

(30) Foreign Application Priority Data
Dec. 23, 2010 (US) 61426913

(51) Int. Cl. A61B 3/10

(52) U.S. Cl. CPC A61B 3/10
USPC 348/78

(57) ABSTRACT

Exemplary embodiments of the present disclosure can provide a retinal camera. The exemplary camera can include a body, at least one optical sensor which is (i) provided on the body, and (ii) configured to receive light directly from a lens of an eye, and a processing arrangement configured to generate an image based on the light received directly from the lens of the eye.

### CROSS-REFERENCE TO PRIOR APPLICATIONS

The present application claims priority to U.S. Provisional Application Ser. No. 61/426,913, filed on Dec. 23, 2010, which is hereby incorporated by reference herein in its entirety.

### FIELD OF THE DISCLOSURE

Exemplary embodiments of the present disclosure relate to medical imaging, and more specifically to a lensless imaging method and apparatus for imaging an eye.

### BACKGROUND INFORMATION

A retinal or fundus camera can be a specialized low power microscope with an attached camera typically used to image the retina, the neurosensory tissue in eyes which can translate the optical images seen by people into the electrical impulses our brain understands. Various retinal cameras have been developed by merely adding a camera to complicated equipment that was originally designed to be used by a human, e.g., an examiner or a physician, looking into the eye of a patient. Retinal cameras merely replaced the examiner's eye in these systems with a camera. The complicated optical systems, however, were not simplified.

Such retinal cameras can be used for retinal imaging for screening diabetes, high blood pressure, and other diseases associated with retinopathies. Retinal cameras are typically described by the angle of view, e.g., the optical angle of acceptance of the lens. For example, an angle of about 30°, which is generally considered the normal angle of view, can create a film image about 2.5 times larger than actual size. Wide angle retinal cameras can typically capture images between about 45° and 140°, and provide a proportionately smaller retinal magnification.

Conventional retinal cameras typically have an objective lens and typically require a complex alignment system to obtain the image. These cameras generally operate by directing a flash of light out through an objective lens, through the patient's pupil and onto the retina. The light that reflects from the retina passes back through the objective lens before being directed onto a digital charge-coupled device (CCD), which captures the image. Some recently developed cameras can use optical coherence tomography (OCT), which can produce high-resolution three-dimensional (3D) images. Further, prior to imaging, the patient's pupil is typically dilated with drops to prevent constriction of the pupil from the light flash. However, several non-mydriatic cameras which do not require the dilation have been developed. The procedure of imaging the patient's retina typically takes about 5-10 minutes.

As noted, retinal cameras can be used for retinal imaging for screening diabetes, high blood pressure and other diseases associated with retinopathies. However, appropriate screening for diabetic patients or for open angle glaucoma is still lacking despite certain progress made in imaging technologies. An affordable apparatus and method which can acquire images and transmit such images electronically to a diagnostic center, facilitating real time or near-real time feedback can provide improved levels of follow-up care that is typically not currently available. For example, with respect to a diabetic retinopathy, an early stage follow-up at about 12 months interval can typically be appropriate. For a proliferative situation, however, an immediate follow-up at a higher level center may be appropriate. Further, asking a patient to go for a follow-up only when necessary can greatly improve compliance and the outcome, as well as the cost of health care.

Further, existing cameras can be prohibitively expensive. For example, conventional midriatic or non-midriatic cameras can presently cost in the range of about $20,000 to $40,000. Further, such cameras can require obtaining multiple field images (e.g., 9) as separate pictures using very bright flashes. Existing non-midriatic, large field option (e.g., Optomap) can utilize lasers, and is typically extremely expensive (e.g., over $100,000). Further, these cameras typically have colors specified at different spectrums, typically requiring special interpretation and making it less popular with many clinicians.

Thus, it may be beneficial to address and/or overcome at least some of the deficiencies of the prior approaches, procedures and/or systems that have been described herein above.

### SUMMARY OF EXEMPLARY EMBODIMENTS

According to an exemplary embodiment of the present disclosure, a retinal camera can be provided. The exemplary camera can include a body, at least one optical sensor which is, e.g., (i) provided on the body and (ii) configured to receive light directly from a lens of an eye, and a processing arrangement configured to generate an image based on the light received directly from the lens of the eye. The body can include a substantially semi-spherical shape. The optical sensor can include a charge-coupled device (CCD) and/or a complementary metal-oxide-semiconductor (CMOS) sensor, and the optical sensor can include a flat chip and/or an array of optical sensors. The optical sensor can be situated on the body so as to obtain a wide angle image of the eye or a narrow-angle image of the eye. Additionally, the camera can include an adjustable flexible positioning arrangement and/or at least one actuator associated with the optical sensor configured to focus the camera. Further, the path of the light from the lens to the optical sensor can be absent of optical elements, which can include an objective lens, a pinhole, a slit, and/or an optical arrangement having an aperture.

Accordingly to another exemplary embodiment, the camera can include at least one illumination element disposed on the body, and the camera can also include a corrective lens configured to correct solely refractive defects of the lens of the eye.

According to yet another exemplary embodiment of the present disclosure, a method for imaging an eye can be provided. The exemplary method can include a receipt of light directly from a lens of an eye using at least one optical sensor positioned on a body, and a generation of an image based on the light received directly from the lens of the eye. The optical sensor can include a charge-coupled device (CCD) and/or a complementary metal-oxide-semiconductor (CMOS) sensor, and can include a flat chip or an array of optical sensors. Further, a path of the light from the lens of the eye to the optical sensor can be absent of optical elements, which can include an objective lens, a pinhole, a slit, and/or an optical arrangement having an aperture.

Accordingly to another exemplary embodiment, the exemplary method can include receiving the optical sensor(s) in the body, and can also include positioning the body so as to place the optical sensor(s) in direct communication with a lens of the eye. The exemplary method can also include focusing the image by moving the spherical body and/or the optical sensor(s).

These and other objects, features and advantages of the exemplary embodiment of the present disclosure will become apparent upon reading the following detailed description of the exemplary embodiments of the present disclosure, along with the claims which are provided herewith.

### BRIEF DESCRIPTION OF THE DRAWINGS

Further objects, features and advantages of the present disclosure will become apparent from the following detailed description taken in conjunction with the accompanying drawings showing illustrative embodiments of the present disclosure, in which:

FIG. 1 is a frontal cross-sectional view of an exemplary retinal camera according to an exemplary embodiment of the present disclosure;

FIG. 2 is a side cross-section illustration of the exemplary retinal camera of FIG. 1 represented relative to an eye according to the exemplary embodiment of the present disclosure;

FIG. 3 is a flow diagram of an exemplary method according to an exemplary embodiment of the present disclosure; and

FIG. 4 is a block diagram of an exemplary system in accordance with certain exemplary embodiments of the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative embodiments and the claims which are provided herewith. It is intended that changes and modifications can be made to the described exemplary embodiments without departing from the true scope and spirit of the subject disclosure.

### DETAILED DESCRIPTION OF EXEMPLARY EMBODIMENTS

The eye can be viewed as a perfect optical system. In addition to providing the ability to see, the structure of the eye in reverse can be used as a projection system. According to certain exemplary embodiments of the present disclosure, a camera can be provided that can utilize the structure of an eye to eliminate various optical features required in existing retinal cameras. For example, the exemplary camera according to certain exemplary embodiments of the present disclosure can utilize the lens of an eye as a lens, thus eliminating the need for an optical element such as an objective lens, an aperture, a slit, and the like. Accordingly, an affordable, cost-effective lensless retinal camera according to certain exemplary embodiments of the present disclosure can provide large field images that is preferably not dependent on the size of the pupils (e.g., does not require dilating the pupils). Further, the exemplary camera can decrease exposure to radiation used during imaging, for example, by decreasing the time of exposure to the radiation when compared to conventional cameras. Exemplary cameras according to certain exemplary embodiments of the present disclosure can be used on patients that are emmetropic, myopic, and hypermetropic. The exemplary camera can be used, for example, for normal retinal imaging in ophthalmology and optometry offices, and also for screening, diagnosis, and monitoring of retinopathies or diseases with retinal symptoms, including diabetes/diabetic retinopathy, high blood pressure/hypertensive retinopathy, macular degeneration, glaucoma, retinal detachment, and other diseases for different retinopathies.

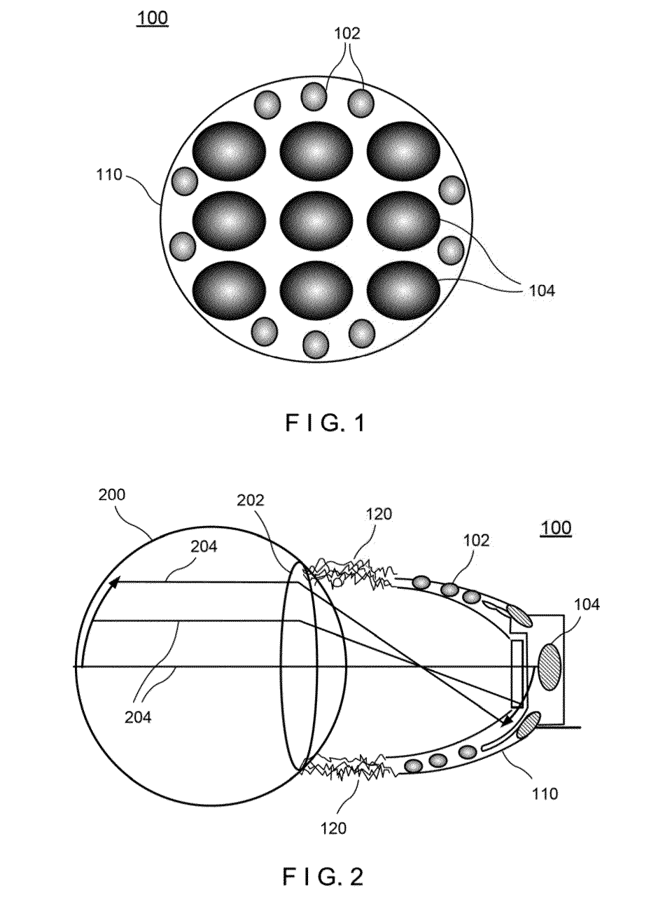

FIG. 1 shows a frontal view of an exemplary camera 100 according to certain exemplary embodiments of the present disclosure. As shown in FIG. 1, camera 100 can have one or more illumination elements 102 disposed on a body 110. Further, camera 100 can include one or more image sensors 104 disposed on the body 110.

The exemplary camera 100 according to certain exemplary embodiments of the present disclosure can have a semi-spherical, elongated semi-spherical, or other similarly shaped body 110. The body 110 can be made of any suitable material, such as plastics, metals, synthetics, polymers, natural materials, and the like. As shown in FIG. 2, the body 110, for example, can have a cross-sectional U-shape, and may have an opening at a front end that can allow light (exemplary light rays illustrated as 204) to enter the body 110. In an exemplary embodiment, the camera 100 can be mounted on to a glasses frame, or in a tube, a frame, a support system, or the like.

The exemplary camera can include an image sensor 104 having, e.g., a photosensitive material provided on an inner surface of the semi-spherical body 110, as shown in FIG. 2. The photosensitive material can include a charge-coupled device (CCD) or a complementary metal-oxide semiconductor (CMOS) chip, or other types of photo/image/optical sensors. The image sensor 104 can include a flat chip for a narrow angle image, or can include an array of chips for obtaining wide-angle images. In an exemplary camera having an array of chips, interpolation procedures can be used to construct the wide-angle image from the array of chips.

Additionally, as shown in FIGS. 1 and 2 and as noted above, the exemplary camera 100 can include certain illumination elements or arrangements 102, such as light-emitting diodes (LEDs), lasers, or the like, disposed on the inner surface of the body 110. In one exemplary embodiment, the image sensors 104 can be arranged towards the center of the body 110, as shown in FIG. 1, and the illumination elements 102 can be arranged around the periphery of the image sensors 102.

Unlike conventional cameras, the exemplary camera 100 can exclude an objective lens, or other optical elements or features (e.g., a pinhole, an aperture, a slit, or the like). Instead of using complicated device optics, the exemplary camera can utilize the optics (shown as 202 in FIG. 2)of the human eye (shown as 200 in FIG. 2) to capture an image. The exemplary configuration of the exemplary camera 100 can facilitate using the lens 202 of the eye 200 to serve as lens of the camera 100, obviating the need for an additional optical system. Accordingly, light (e.g., 204) can be projected onto the image sensors directly from the lens 202 of an eye 200 that is being imaged. Thus, optical elements or features can be excluded from the path that the light travels from the lens 202 of the eye 200 to the image sensors 104 of the exemplary camera 100 that focus or otherwise alter the light 204. In this manner, the exemplary camera can be in direct communication with the eye 200 that is being imaged, so that light emanating from the lens 202 of the eye 200 can be received directly by the image sensors 104.

Since the exemplary camera 100 can preferably exclude an objective lens, focusing the exemplary camera 100 can be performed by moving the camera 100 and/or the image sensors 104 relative to the eye of the patient. In an exemplary embodiment, the camera 100 can include a positioning element 120, such as, e.g., a flexible cone (as shown in FIG. 2) and/or actuators, which can facilitate moving the camera 100 and/or the image sensors 104 so as to position the camera 100 and/or the image sensors 104 at a desired distance relative to the eye. For example, if the distances from an object to the lens (e.g., the distance from the retina to the lens of the eye) and from the lens to the image sensors 104 (e.g., the distance from the lens of the eye to the image sensors) are D1 and D2, respectively, for a lens of negligible thickness, in air, the distances can be related by the thin lens formula : 1/D1+1/D2=1/f. Accordingly, if an object, e.g., the retina, is placed at a distance D1 along the axis in front of a positive lens. e.g., the lens of the eye, of focal length f, a screen, e.g., the image sensors, placed at a distance D2 behind the lens will have a sharp image of the object projected onto it, as long as D1>f (if the lens-to-screen distance D2 is varied slightly, the image will become less sharp).

According to an exemplary embodiment of the present disclosure, the camera 100 can include the positioning element 120 at the edge of the opening, such as, e.g., a flexible cone, to move and position the entire camera relative to the eye. The flexible cone can be made of any flexible material such as, e.g., silicone. In another exemplary embodiment, the image sensors 104 can include actuators that can move the individual image sensors 104 relative to the body 110 of the camera 100 instead of or in addition to moving the entire camera 100. In a further alternative exemplary embodiment of the present disclosure, the camera 100 can include (i) a positioning element 120 at the edge of the opening and (ii) the actuators associated with the image sensors.

According to still another exemplary embodiment of the present disclosure, the exemplary camera 100 can be used in conjunction with a corrective lens. The corrective lens preferably does not act as an objective lens/optical system having apertures and optical stops for the exemplary camera. Instead, the corrective lens can simply correct only the refractive status of the eye, e.g., correct the vision of the patient. For example, corrective lenses can be used on patients who are hypermetropic so as to correct the patients to be either emmetropic or myopic. In exemplary embodiments, corrective lenses having a diopter strength of, e.g., +10, +20, or the like can be used. Further, the corrective lens can be a contact lens, a glasses lens, or the like. Further, the operation of the exemplary camera would preferably not change whether or not a corrective lens is worn by the patient.

The exemplary camera 100 according to certain exemplary embodiments of the present disclosure can provide wide angle and high resolution images that can be well suited to digital processing. Further, the exemplary camera 100 can obtain the wide angle/wide filed of view images of the retina without being dependent on the pupil size (e.g., the exemplary camera can obtain wide angle images even with a very small pupil). The exemplary camera 100 can provide a small footprint that can increase the appeal of taking photos for screening applications. Further, the configuration of the exemplary camera 100 can facilitate the manufacturing thereof at a lower cost, and provide faster image acquisition time than current retinal cameras, and the size and operability can also make it well-suited for use in telemedicine. In addition to being used in modern clinics, the exemplary camera 100 can be used in various other applications, such as in screening programs in developing areas of the world because it can be inexpensive, small, and easy to operate.

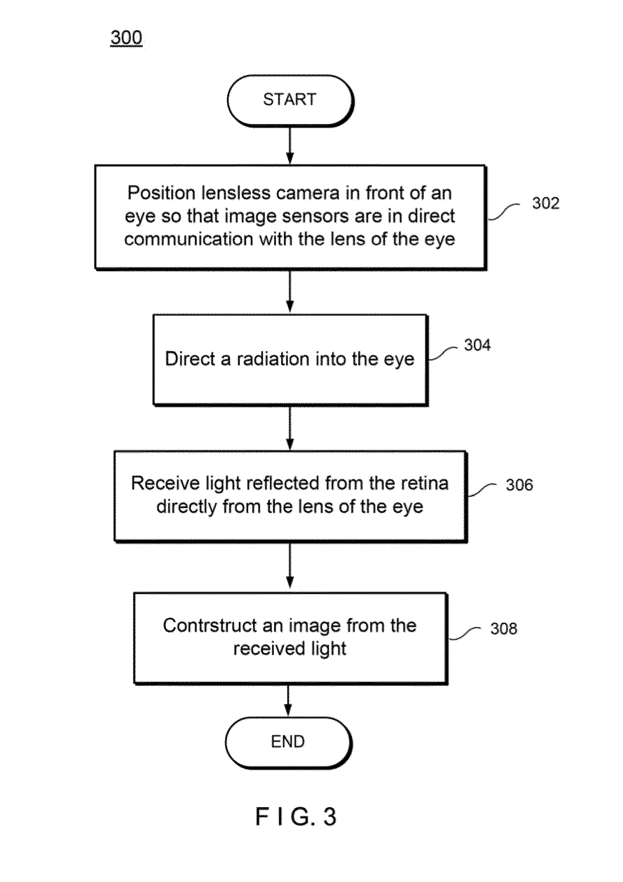

FIG. 3 shows an exemplary flow diagram of an exemplary method 300 in accordance with an exemplary embodiment of the present disclosure. In the exemplary method 300, an exemplary lensless camera in accordance with certain exemplary embodiments of the present disclosure can be positioned in front of the eye to be imaged so that the image sensors of the camera can be in direct communication with the lens of the eye (procedure 302). Next, a radiation (e.g., light) can be directed into the eye (procedure 304). Then, the light reflected off the retina can be received by the optical sensors directly and unaltered from the lens of the eye (procedure 306). This received light can then be used to construct an image (procedure 308). Optionally, the camera or the optical sensors can be moved relative to the eye so as to focus the camera. Further, a corrective lens can also be used in conjunction with the camera and the eye.

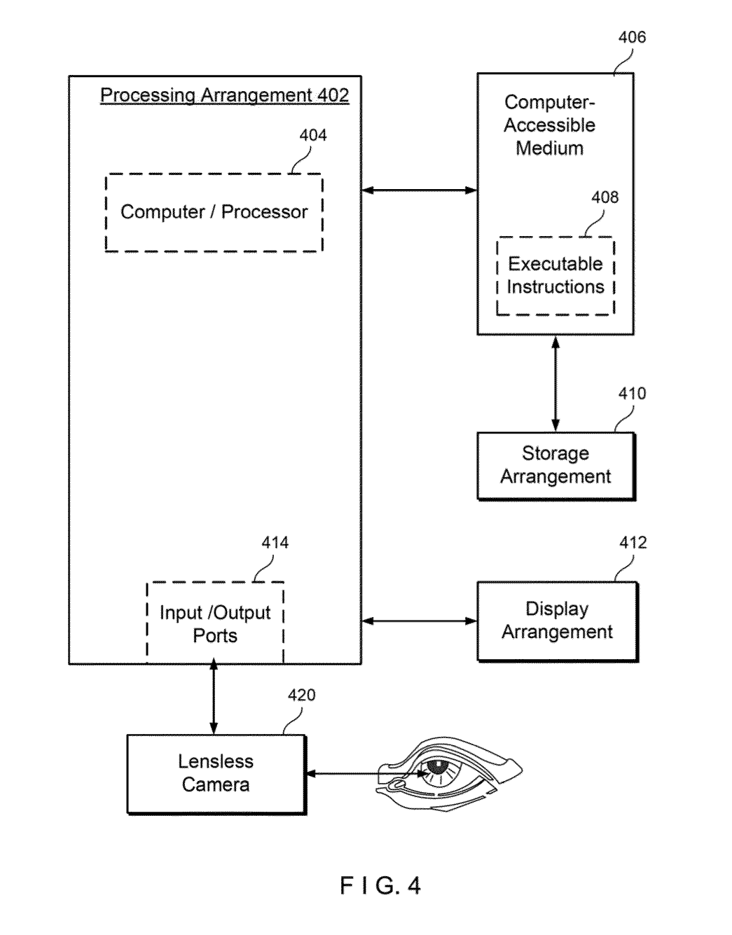

FIG. 4 shows an exemplary block diagram of an exemplary embodiment of a system according to the present disclosure. For example, exemplary procedures in accordance with the present disclosure described herein can be performed by an imaging arrangement 420 (e.g., the exemplary lensless camera 100) and a processing arrangement and/or a computing arrangement 402. Such processing/computing arrangement 402 can be, e.g., entirely or a part of, or include, but not limited to, a computer/processor 404 that can include, e.g., one or more microprocessors, and use instructions stored on a computer-accessible medium (e.g., RAM, ROM, hard drive, or other storage device).

As shown in FIG. 4, e.g., a computer-accessible medium 406 (e.g., as described herein above, a storage device such as a hard disk, floppy disk, memory stick, CD-ROM, RAM, ROM, etc., or a collection thereof) can be provided (e.g., in communication with the processing arrangement 402). The computer-accessible medium 406 can contain executable instructions 408 thereon. In addition or alternatively, a storage arrangement 410 can be provided separately from the computer-accessible medium 406, which can provide the instructions to the processing arrangement 402 so as to configure the processing arrangement to execute certain exemplary procedures, processes and methods, as described herein above, for example.

Further, the exemplary processing arrangement 402 can be provided with or include an input/output arrangement 414, which can include, e.g., a wired network, a wireless network, the internet, an intranet, a data collection probe, a sensor, etc. As shown in FIG. 4, the exemplary processing arrangement 402 can be in communication with an exemplary display arrangement 412, which, according to certain exemplary embodiments of the present disclosure, can be a touch-screen configured for inputting information to the processing arrangement in addition to outputting information from the processing arrangement, for example. Further, the exemplary display 412 and/or a storage arrangement 410 can be used to display and/or store data in a user-accessible format and/or user-readable format.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. In addition, all publications and references referred to above can be incorporated herein by reference in their entireties. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly being incorporated herein in its entirety. All publications referenced are incorporated herein by reference in their entireties.

What is claimed is:

1. A retinal camera, comprising:
a body;
at least one optical sensor which is (i) provided on the body, and (ii) configured to receive light directly from a lens of an eye; and
a processing arrangement configured to generate an image based on the light received directly from the lens of the eye.

2. The camera of claim 1, wherein a path of the light from the lens of the eye to the at least one optical sensor is absent of optical elements.

3. The camera of claim 2, wherein the optical elements include at least one of an objective lens, a pinhole, a slit, or an optical arrangement having an aperture.

4. The camera of claim 1, further comprising at least one illumination element disposed on the body.

5. The camera of claim 1, wherein the at least one optical sensor includes at least one of a charge-coupled device (CCD) or a complementary metal-oxide-semiconductor (CMOS) sensor.

6. The camera of claim 1, wherein the at least one optical sensor includes at least one of a flat chip or an array of optical sensors.

7. The camera of claim 1, further comprising at least one actuator associated with the at least one optical sensor and configured to focus the camera.

8. The camera of claim 1, further comprising an adjustable flexible positioning arrangement configured to focus the camera.

9. The camera of claim 1, further comprising a corrective lens configured to correct solely refractive defects of the lens of the eye.

10. The camera of claim 1, wherein the at least one optical sensor is situated on the body so as to obtain a wide-angle image of the eye.

11. The camera of claim 1, wherein the at least one optical sensor is situated on the body so as to obtain a narrow-angle image of the eye.

12. The camera of claim 1, wherein the body has a substantially semi-spherical shape.

13. The camera of claim 1, wherein the light received from the lens of the eye is reflected from a retina of the eye prior to passing through the lens of the eye.

14. A method for imaging an eye, comprising:
receiving light directly from a lens of an eye using at least one optical sensor positioned on a body; and
generating an image based on the light received directly from the lens of the eye.

15. The method of claim 14, further comprising receiving at least one optical sensor in the body.

16. The method of claim 14, further comprising positioning the body so as to place the at least one optical sensor in direct communication with the lens of the eye.

17. The method of claim 14, wherein a path of the light from the lens of the eye to the optical sensor is absent of optical elements.

18. The method of claim 17, wherein the optical elements include at least one of an objective lens, a pinhole, a slit, or an optical arrangement having an aperture.

19. The method of claim 14, further comprising focusing the image by moving at least one of the spherical body or the at least one optical sensor.

20. The method of claim 14, wherein the at least one optical sensor includes at least one of a charge-coupled device (CCD) or a complementary metal-oxide-semiconductor (CMOS) sensor.

21. The method of claim 14, wherein the at least one optical sensor includes at least one of a flat chip or an array of optical sensors.

22. The method of claim 14, wherein the light received from the lens of the eye is reflected from a retina of the eye prior to passing through the lens of the eye.
